(19) United States
(12) Patent Application Publication
(10) Pub. No.: US 2008/0319454 A1
(43) Pub. Date: Dec. 25, 2008

(54) METHOD FOR USING A THREAD FOR HAIR REMOVAL AND SAME

(76) Inventor: Samir Figuigui, Oak Lawn, IL (US)

Correspondence Address: MARTIN E. JERISAT, 3300 CHASE PLAZA, 10 S. LaSalle Street, Chicago, IL, 60603, US

(21) Appl. No.: 11/768,148

(22) Filed: Jun. 25, 2007

(51) Int. Cl. A61B 17/50

(52) U.S. Cl. 606/133; 606/131

(57) ABSTRACT

A method for using a thread for hair removal and a hair removal method are provided here. The method includes the act of forming an entrapment to trap the hair that needs to be removed. The entrapment may then be pulled away from the skin to lift the hair out of its follicle. A topical anesthetic solution is applied to the skin before hair removal in order to alleviate any possible irritation of the skin.

### BACKGROUND OF THE INVENTION

The present invention relates to a method for using a thread for hair removal and a hair removal method. The current methods of removing hair, such as waxing and tweezing, have several disadvantages. Some of these methods peel off layers of skin and leave the skin sagging. Other methods leave the skin red and irritated. Other methods do not pull out specific individual single hairs. Other methods cause hah strong hair re growth.

Accordingly, it would be advantageous to provide a method that does not peel off layers of skin and does not leave the skin sagging after the removal of the hair. It would also he advantageous to provide a method capable of picking up the finest hah: from the skin surface arid that does not cause strong hair re growth.

### SUMMARY OF THE INVENTION

The objects mentioned above, as well as other objects, are solved by the present invention, which overcomes disadvantages of prior hair removal methods, while providing new advantages not believed associated with such systems.

The method involves the use of a thread to remove hair from its follicle. In an embodiment, the thread may be twisted back and fourth around the hair that needs to be removed. The twisting of the hair may trap the hair. The thread may be pulled away from the skin in order to lift the hair out of its follicle. A topical anesthetic solution may be applied to the skin before hair removal in order to lessen any possible irritation of the skin doe to the forceful removal of the hair. A disinfectant may also be applied to the skin.

The thread may be twisted to form a substantially curvilinear lobe. The lobe may further be twisted to form a knot. The lobe may be used to trap the hair that needs to be removed. The lobe may then be pulled away from the skin in order to lift the hair out of its follicle.

In an embodiment, the thread may be formed into one and another substantially curvilinear lobe. The lobes may be of same or different areas. One of the lobes may be applied to the hair that needs to be removed in order to trap the hair. The other lobe may then be expanded to squeeze the hair in the other lobe out of its follicle.

### BRIEF DESCRIPTION OF THE DRAWINGS

The novel features which are characteristic of the invention are set forth in the appended claims. The invention itself however, together with further objects and attendant advantages thereof, will be best understood by reference to the following description taken in connection with the accompanying drawings. The drawings illustrate currently preferred embodiments of the present invention. As further explained below, it will be understood that other embodiments, not shown in the drawings, also fall within the spirit and scope of the invention.

FIG. 1 shows a user holding a thread;

FIG. 2 shows the formation of a lobe;

FIG. 3 is shows entrapment of the hair;

FIG. 4 shows the removal of the hair;

FIG. 5 shows another embodiment of the invention;

FIG. 6 shows the formation, of two lobes;

FIG. 7 shows twisting of the thread to sharpen the lobes and

FIG. 8 shows the act of hair removal; and

FIG. 9 shows an alternative embodiment of the invention.

### DETAILED DESCRIPTION OF THE PREFERRED EMBODIMENTS

Set forth below is a description of what are currently believed to be the preferred embodiments and/or best examples of the invention claimed. Future and present alternatives and modifications to these preferred embodiments are contemplated. Any alternatives or modifications which make insubstantial changes in function, in purpose, in structure or in result are intended to be covered by the claims of this patent.

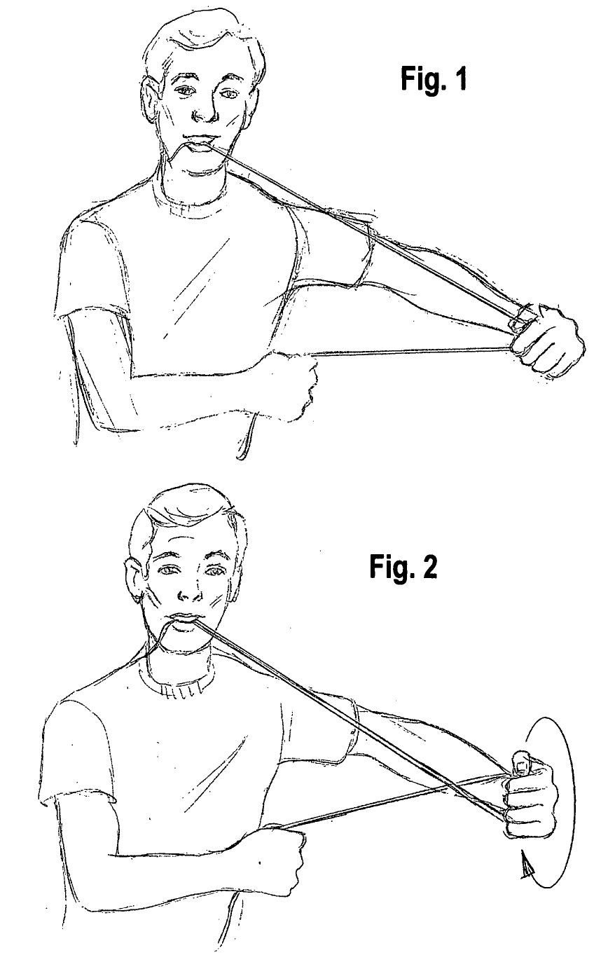

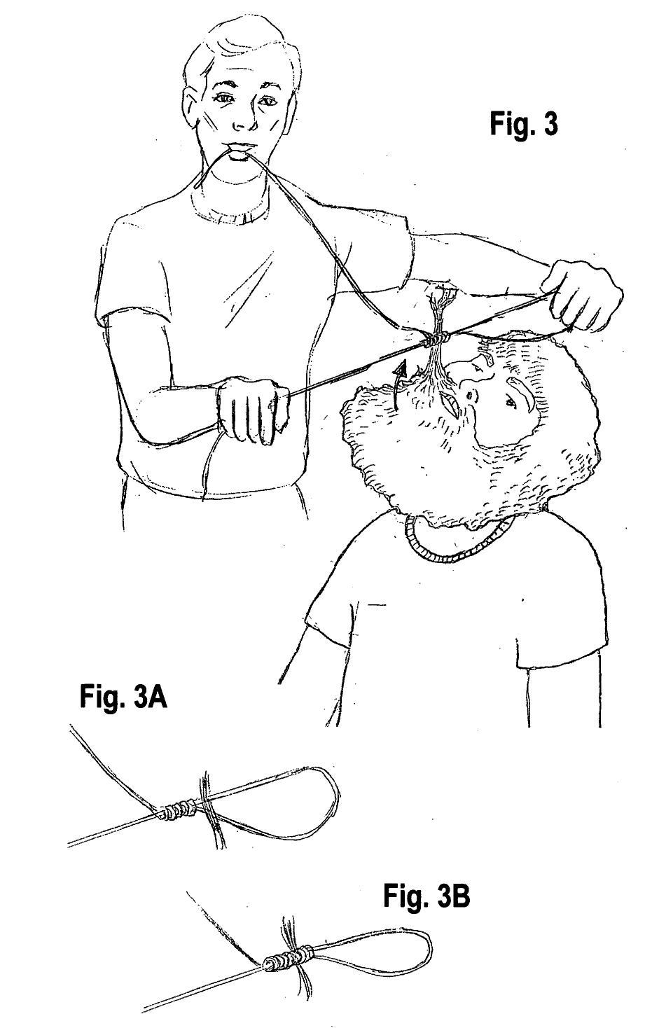

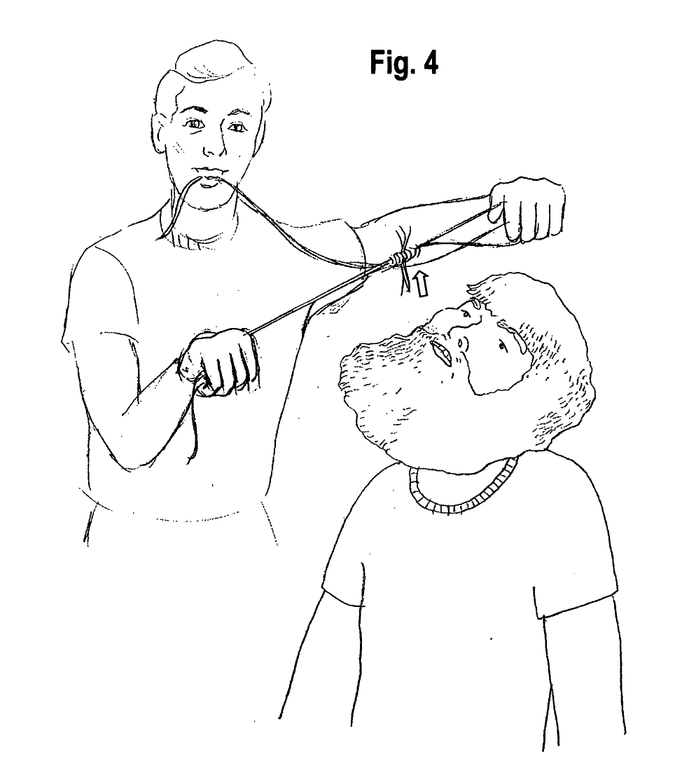

Referring first to FIGS. 1-4, FIG. 1 shows one embodiment of the present invention. A user 10 holds a thread 15 between the mouth 11 and his hands 12 and 13. As shown in FIG. 2, user 10 may twist thread 15 in order to form one lobe 16. User 10 may also twist thread 15 several times in order to form a knot 17, as shown in FIG. 3, to assist in the hair removal process. FIG. 3 also shows that lobe 16 may be wrapped about the hair 19 in order to trap hair 19. FIG. 4 shows user 10 squeezing hair 19 between knot 17 and lobe 16. Thread 15 may then be pulled away from the skin in order to lift hair 19 out of its follicle, not shown.

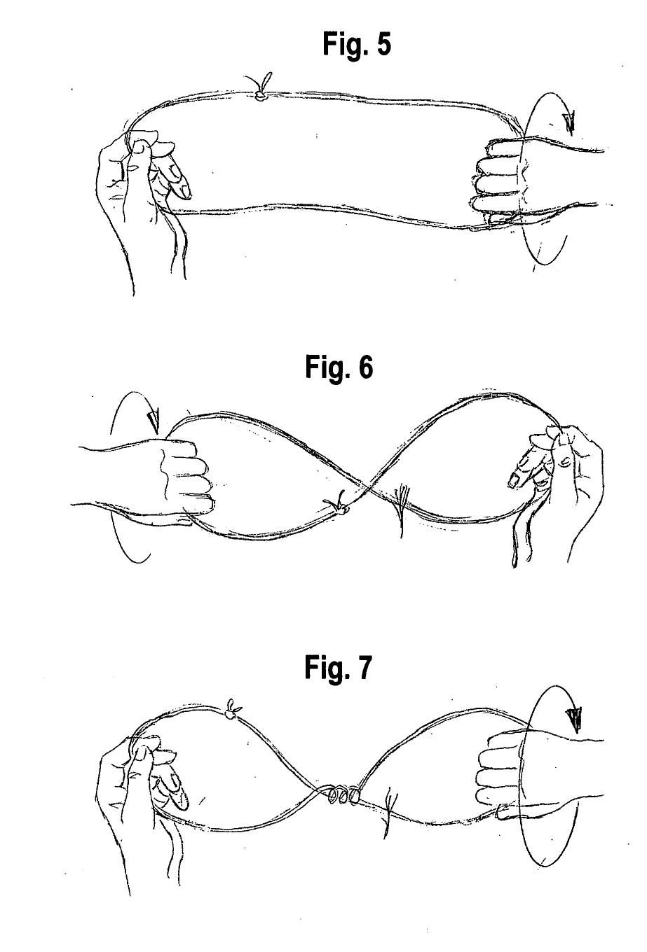

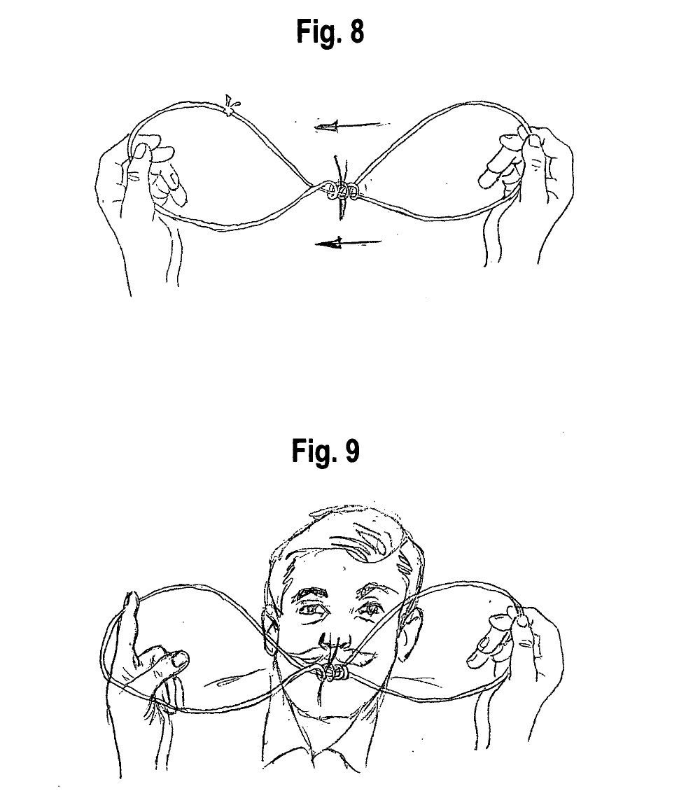

Referring now to FIGS. 5-8, FIG. 5 shows another embodiment of the present invention. Thread 15 may be held between the hands 12 and 13. FIG. 6 shows that thread 15 may be twisted to form an entrapment, such as two lobes 21 and 22 of similar or different sizes. One of the lobes 19 may be wrapped around hair 19 on skin area 20. FIG. 7 shows that thread 15 may be twisted several times to form the knot 17A somewhere between lobes 21 and 22. FIG. 8 illustrates one method for hair removal. Lobe 22 may be expended while holding lobe 21 in order to squeeze hair 19 out of its follicle. An anesthetic topical solution may be applied to skin area 20 in order to alleviate any possible irritation to the skin due to the forceful removal of the hair. A disinfectant solution may also be applied to skin area 20 after hair removal. In an embodiment, a growth inhibiting solution may also be applied to skin area 20 after hair removal.

Referring now to FIG. 9, the figure shows an alternative embodiment of the present invention. User 10 may form an entrapment using thread 15 into two lobes 23 and 24 in order to trap hair 19. User 10 may tie one free end of thread 15 to one of the fingers of hand 12 or 13 and tie another free end of thread 15 to another finger of same hand 12, thereby forming two lobes 23 and 24. User 10 may also twist thread 15 several times in order to form a knot 25. Hair removal may be done by expanding one of the lobes 23, for example, using finger of hand 12 in order to squeeze hair 19 between knot 25 and the fingers of hand 13 and thereby pulling hair 19 out of its follicle.

It will be understood that various modifications to the preferred embodiment disclosed above may be made. The above description is not intended to limit the meaning of the words used in the following claims that define the invention. Rather, it is contemplated that future modifications in structure, function or result will exist that are not substantial changes and that all such insubstantial changes are intended to be covered by the following claims.

I claim:

1. A method for hair removal, comprising the acts of providing a thread having two free ends; tying one end of the thread to a finger of one hand; tying the other end to another finger of the one hand; twisting the thread back and fourth along the hair that needs to be removed; trapping the hair and pulling the thread away from the skin to lift the hair out of its follicle.

2. The method of claim 1 further comprising the act of applying a disinfectant to the skin area after hair removal.

3. The method of claim 1 further comprising the act of applying a growth inhibition solution to the skin area after hair removal.

4. The method of claim 1 further comprising the act of applying a topical anesthetic to the skin area before removing the hair.

5. A method for removing hair, comprising the acts of applying a topical anesthetic solution to the skin area where the hair needs to be removed; providing a thread; forming a lobe; trapping the hair in the lobe and pulling the lobe away from the skin to lift the hair out of its follicle.

6. The method of claim 5 further comprising the act of applying a disinfectant to the skin area after hair removal.

7. A method for using a thread for hair removal, comprising the acts of forming an entrapment with the thread; entrapping the hair; and pulling the entrapment away from the skin to lift the hair out of its follicle.

8. The method of claim 7 wherein the act of entrapment includes the act of creating at least one substantially curvilinear lobe to capture the hair that needs to be removed.

9. The method of claim 7 wherein the act of entrapment includes the act of creating a lobe and another substantially curvilinear lobe to capture the hair that needs to be removed in the other lobe.

10. The method of claim 9 wherein the act of pulling the entrapment away from the skin includes the act of expanding the one lobe to squeeze the hair in the other lobe out of its follicle.

11. The method of claim 7 farther including the act of applying a topical anesthetic to the skin area before hair removal.

12. The method of claim 7 further including the act of applying a disinfectant to the skin area after hair removal.
